(19) United States
(12) Patent Application Publication
(10) Pub. No.: US 2019/0021961 A1
(43) Pub. Date: Jan. 24, 2019

(54) WATER-IN-OIL EMULSION

(71) Applicant: Dr. August Wolff GmbH & Co, KG Arzneimittel, Bielefeld (DE)

(72) Inventors: Christoph Abels, Bielefeld (DE); Ulrich KNIE, Bielefeld (DE)

(21) Appl. No.: 16/066,923

(22) PCT Filed: Jan. 13, 2017
(86) PCT No.: PCT/EP2017/050619

§ 371 Date: Jun. 28, 2018

(30) Foreign Application Priority Data
Jan. 15, 2016 (EP) 16151515.0

(51) Int. Cl. A61K 8/06; A61K 8/365; A61K 8/37; A61K 8/60; A61K 8/31; A61Q 19/00; A61Q 19/08; A61K 9/107; A61K 9/06; A61K 9/08; A61K 47/12; A61K 47/26; A61K 47/14

(52) U.S. Cl. CPC A61K 8/064; A61K 8/365; A61K 8/375; A61K 8/60; A61K 8/31; A61Q 19/007; A61K 47/14; A61K 9/107; A61K 9/06; A61K 9/08; A61K 47/12; A61K 47/26; A61Q 19/08

(57) ABSTRACT

The present invention relates to a water-in-oil emulsion and its use as a skin care product, in particular as a cosmetic and as a (dermatologic) medicament for aged skin. The emulsion contains a disperse/internal aqueous phase comprising an α-hydroxy-C₂-C₆-carboxylic acid, a continuous/external oil phase and an emulsifier system, comprising polyglyceryl-3-polyricinoleate, sorbitan oleate and sucrose polystearate, wherein the emulsion has a pH of 1 to 5.

The present invention relates to a water-in-oil emulsion and use thereof as a skin care product, in particular as a cosmetic and as a (dermatologic) pharmaceutical product for older skin in particular.

The pH of skin is of crucial importance for the physiological functions of skin. The pH of older skin as well as that of diseased skin is known to be higher in comparison with that of young and healthy skin (i.e., the acidity at the skin surface and/or in the stratum corneum is lowered) and therefore physiological skin functions may be impaired. To counteract this problem, it has been proposed that skin care products be formulated with a lower pH for older skin. It is assumed that this would normalize the age-related increase in the pH of skin and allow the physiological skin functions to be maintained (Schreml et al., EMJ Dermatol. 2014; 2:86-94).

Since older skin also loses its moisture content more easily than young skin, ingredients that release moisture are added to skin care products for older skin to prevent dehydration, i.e., drying, of skin. One measure that is especially suitable is to formulate the products in the form of water-in-oil (W/O) emulsions because these usually have a much longer-lasting moisture-releasing effect on skin than oil-in-water emulsions (O/W) (Daniels et al., Galenik der Dermatika Grundlagen, Eigenschaften, Freisetzung [Galenical Principles of Dermatologic Preparations Principles, Properties, Release]; J. Dtsch Dermatol Ges 2007; 5:367-383).

However, W/O emulsions have some fundamental disadvantages in comparison with O/W emulsions. Theoretically, for example, they release the protons (Fr), that are present in the internal aqueous phase and that ultimately influence the pH of skin, more slowly than O/W emulsions in which the protons are present in the external/continuous phase. Consequently, an O/W emulsion should be much more suitable than a W/O emulsion for the above-mentioned potential skin care products, which have a lower pH for treatment of older skin, with which a rapid and definite reduction in pH is desired. In addition, W/O emulsions are usually less stable in comparison with O/W formulations, in particular at a low pH. The problem lies in the adequate stability of traditional emulsifier systems under acidic conditions, among other things. Many emulsifiers are not resistant to hydrolysis at a low pH, which, first of all, reduces the emulsifier effect and secondly can result in unwanted by-products.

In a study published recently, it was found that in healthy patients, the pH of skin could be reduced significantly by treatment with a W/O emulsion (pH 4) two to four times daily for a period of 28 days. However, the study did not reveal the precise composition of the W/O emulsion. Furthermore, the stability of the W/O emulsion was not taken into account in this study, nor was a direct comparison with an O/W emulsion made with regard to reducing the pH of skin (Behm et al., Skin Pharmacol Physiol 2015; 28:290-295).

There is therefore a demand for W/O emulsions that will be stable even at a low pH and can lower the pH of skin quickly and decisively, while being suitable as a skin care product that will release moisture to the skin over a long period of time for treatment of older or diseased skin.

The object on which the present invention is based thus lies in providing a W/O emulsion that will be stable in the acidic pH range, while effectively correcting the age-related or disease-induced disturbance in skin function.

This object has surprisingly been achieved according to the subject matter of the present invention.

The present invention relates to a water-in-oil emulsion (also referred to in general as a W/O emulsion) that contains a dispersed (i.e., internal) aqueous phase, a continuous (i.e., external) oil phase and a specific emulsifier system. One essential ingredient of the emulsion according to the invention is also an □-hydroxy-C₂-C₆-carboxylic acid, which is present primarily in the aqueous phase due to its solubility, and which adjusts the pH of the emulsion according to the invention to 1 to 5. The specific emulsifier system comprises essentially three ingredients, i.e., polyglyceryl-3-polyricinoleate, sorbitan oleate and sucrose polystearate as well as the respective derivatives thereof.

It has surprisingly been found that the W/O emulsion according to the invention has an extremely good stability at a low pH, which is attributed to the specific emulsifier system. At the same time, the pH on the skin (pHss) as well as that in the stratum corneum (pHsc) can be lowered effectively by using the W/O emulsion according to the invention. Therefore, the W/O emulsion according to the invention makes it possible to effectively restore the physiological properties (such as the epidermal barrier functions and barrier integrity) of older and/or diseased skin in particular, in which the acidity at the skin surface and/or in the stratum corneum is reduced. The reduction in pH on the skin and in the stratum corneum according to the invention in comparison with a corresponding oil-in-water emulsion (O/W) is achieved unexpectedly rapidly and distinctly for those skilled in the art, based on their expectations. As a result, it has surprisingly been found that a plurality of medical and cosmetic indications for skin can be treated effectively with the W/O emulsion according to the invention.

The preferred embodiments of the present invention are described in greater detail below.

The aqueous phase of the W/O emulsion according to the invention comprises mainly water as a solvent and optionally additional water-soluble/hydrophilic auxiliary solvents, such as short-chain alcohols (e.g., ethanol, propanol or glycerol). Water is preferably the only solvent in the aqueous phase. The amount of aqueous phase (i.e., only the solvent, not including other ingredients dissolved therein, such as □-hydroxy-C₂-C₆-carboxylic acid) in the entire W/O emulsion preferably ranges from 50 to 80 wt %, especially preferably from 60 to 70 wt %, each based on the total weight of the emulsion.

The oil phase of the W/O emulsion according to the invention may fundamentally contain any lipophilic ingredients with which those skilled in the art are familiar from traditional W/O emulsions. These include, among others, the glycerides (monoglycerides, diglycerides and triglycerides), fatty alcohols, fatty acid esters, hydrocarbons, silicone oils and other synthetic oils and fats. Various vegetable oils and vegetable fats can be mentioned as suitable examples according to the invention, in particular hydrogenated vegetable oils (such as olive oil or sunflower oil), long-chain hydrocarbons (such as paraffin, petroleum jelly, ceresin, ozokerite or isohexadecane) or fatty acid esters (such as cetearyl isononanoate, ethylhexyl stearate, decyl oleate, isopropyl myristate, isopropyl palmitate, glyceryl stearate) and mixtures thereof. Suitable oil phases can be selected from the known materials by those skilled in the art, depending on the desired product. However, according to the invention, the oil phase preferably includes isohexadecane, especially preferably in combination with one or more fatty acid esters (such as cetearyl isononanoate, ethylhexyl stearate and/or decyl oleate) and/or one or more vegetable oils and/or vegetable fats (such as hydrogenated vegetable oils) and/or synthetic oils or fats (such as dicaprylyl ether). In the presence of isohexadecane in the oil phase, an especially high stability of the emulsion is achieved according to the invention. The amount of oil phase (i.e., only the solvent without any other ingredients dissolved therein) in the entire W/O emulsion preferably ranges from 10 to 30 wt %, especially preferably from 15 to 25 wt %, most preferably 18 to 20 wt %, each based on the total weight of the emulsion. The amount of isohexadecane alone in the oil phase is preferably 1 to 10 wt %, especially 5 to 8 wt %, each based on the total weight of the emulsion.

The W/O emulsion according to the invention also contains an □-hydroxy-C₂-C₆-carboxylic acid which serves primarily to adjust the pH of the emulsion and ultimately to lower the pH of the treated skin and/or in the stratum corneum. The W/O emulsion according to the invention has a pH of 1 to 5, preferably from more than 1 to less than 5, especially preferably from 2 to 5, most especially preferably from 2.5 to 4.5, and even more preferably from 3.5 to 4.5. The pH is most preferably 4. According to the invention, the best results with regard to a rapid and definite reduction in the pH on the treated skin as well as in the stratum corneum can be achieved in these pH ranges of the emulsion with at the same time maximum stability of the W/O emulsion.

According to the invention, the □-hydroxy-C₂-C₆-carboxylic acid is preferably a fruit acid selected in particular from the group consisting of glycolic acid, lactic acid, tartaric acid, citric acid, ascorbic acid, malic acid and mixtures thereof. Glycolic acid is especially preferred. If the chain length exceeds C₆, a definite reduction in the penetration of the acid into the stratum corneum is detected, so that the pH-lowering effect of the W/O emulsion on and in the skin is inadequate.

The amount of □-hydroxy-C₂-C₆-carboxylic acid in the W/O emulsion can be adjusted easily by those skilled in the art in accordance with the desired pH. It preferably amounts to 0.5 to 3.0 wt %, preferably 1.0 to 2.0 wt %, □-hydroxy-C₂-C₆-carboxylic acid, based on the total weight of the emulsion.

The W/O emulsion according to the invention may also contain a basic pH-adjusting agent, preferably ammonium hydroxide. The amount depends on the exact pH desired for the emulsion and can be adjusted accordingly by those skilled in the art.

Another ingredient of the W/O emulsion according to the invention is the specific emulsifier system which surprisingly ensures the high stability of the emulsion even at a low pH while at the same time facilitating the rapid and definite reduction in pH on the skin and in the stratum corneum after applying the emulsion to the skin. The emulsifier system comprises essentially polyglyceryl-3-polyricinoleate, sorbitan oleate and sucrose polystearate. According to the invention, derivatives thereof are also included. These three ingredients, considered separately, are known substances which are commercially available as such. Polyglyceryl-3-polyricinoleate (also known as polyglycerol-polyricinoleate; PGPR; E 476; Dermofeel® PR) is a carboxylic acid ester of an oligomeric glycerol with polymeric ricinoleic acid. Sorbitan oleate (also known as sorbitan monooleate; SPAN 80) is the monoester of oleic acid with sorbitan. Sucrose polystearate is a sucrose fatty acid polyester of stearic acid.

The W/O emulsion according to the invention preferably contains 1.0 to 5.0 wt % polyglyceryl-3-polyricinoleate, 2.0 to 10.0 wt % sorbitan oleate and 0.5 to 3.0 wt % sucrose polystearate, each based on the total weight of the emulsion. The inventive emulsion especially preferably contains 1.0 to 2.0 wt % polyglyceryl-3-polyricinoleate, 3.0 to 5.0 wt % sorbitan oleate and 1.0 to 1.5 wt % sucrose polystearate, each based on a total weight of the emulsion. The inventive emulsion most especially preferably contains 1.0 to 1.5 wt % polyglyceryl-3-polyricinoleate, 3.0 to 3.5 wt % sorbitan oleate and 1.0 to 1.5 wt % sucrose polystearate, each based on the total weight of the emulsion. In other words, the weight ratio of polyglyceryl-3-polyricinoleate to sorbitan oleate to sucrose polystearate in the emulsifier system preferably amounts to (1.0-1.5) to (3.0-3.5) to (1.0-1.5) (i.e., (1.0-1.5):(3.0-3.5):(1.0-1.5)), in particular 1.5 to 3.5 to 1.0 (i.e., 1.5:3.5:1.0). The inventive effects with regard to stability and reduction in pH on the skin and in the stratum corneum are achieved especially obviously in the preferred quantity ranges. This is surprising inasmuch as the amounts are low in comparison with the emulsifiers traditionally used. Furthermore, it could not have been foreseen that a particularly great stability could be achieved with, at the same time, effective reduction in skin pH by using the preferred ratios of the three ingredients of the emulsifier system. The emulsifier system preferably consists of polyglyceryl-3-polyricinoleate, sorbitan oleate and sucrose polystearate, i.e., the W/O emulsion according to the invention preferably does not contain any emulsifier other than these three ingredients.

The W/O emulsion according to the inventions may also contain the usual medicinal and cosmetic additives. These include in particular antioxidants, preservatives, stabilizers, emollients and perfume. Antioxidants include, for example, tocopherol or tocopherol acetate, in particular tocopherol. Stabilizers include, for example, magnesium sulfate, acrylate polymers, carbomers, starches and xanthan gum. Suitable emollients include alkyl palmitate compounds (e.g., isopropyl palmitate) as well as paraffin and isoparaffins. Buffer systems such as citric acid and sodium citrate may optionally also be included.

The W/O emulsion according to the invention can be prepared with the help of preparation methods conventionally used for W/O emulsions. For example, the oil phase and the aqueous phase can be heated independently of one another and then mixed together. Before mixing, the additional ingredients (i.e., the emulsifier system, the □-hydroxy-C₂-C₆-carboxylic acid and the other additives, if any) may be added to the oil phase and/or to the aqueous phase. Alternatively, the two phases may also be combined just before the other ingredients are added. The emulsifier system is preferably added before mixing the oil phase, and the □-hydroxy-C₂-C₆-carboxylic acid is usually added to the aqueous phase. Typical temperatures for the preparation include 40° C. to 95° C., preferably 80° C. to 90° C. The mixing takes place in a homogenizer, usually within 5 to 25 minutes at a stirring speed of 300 to 2000 rpm. Then the emulsion is cooled to room temperature.

The W/O emulsion according to the invention may be used as a medicinal product (medicament), preferably as a dermatologic agent, as a medicinal device or as a cosmetic. The inventive use results in a definite improvement in the epidermal barrier functions and the epidermal barrier integrity, an improvement in the appearance of the skin and an increase in the moisture content of the skin. This is associated with an increase in the cohesion of the stratum corneum and homeostasis of the skin barrier and ultimately results in improved protection against infections (microbial diseases).

In medical use, both the therapeutic and prophylactic treatment of skin diseases are included according to the invention. These diseases preferably include microbial skin infections, skin inflammations, raw skin, dry skin, skin irritations, itching, pruritus, allergies, psoriasis, psoriatic arthritis, eczema, scleroderma, atopic dermatitis, contact dermatitis, systemic lupus erythematosus, alopecia areata, acne and susceptibility to contact allergies.

Non-therapeutic (i.e., cosmetic) use includes in particular treatment of cosmetic indications of the skin, in particular selected from raw skin, dry skin, irritated skin, itching and pruritus, as well as prevention of skin infections and reducing the susceptibility of skin to contact allergies.

The W/O emulsion according to the invention may be present in various medical (dermatologic) and/or cosmetic product forms. It is basically suitable for rinse-off products or leave-on products. Leave-on products are preferred. A suitable product form is, for example, a cream, lotion, ointment, makeup, mascara or lipstick, in particular a face cream and a body lotion.

The preferred use of the W/O emulsion according to the invention depends on the specific product form and the medical and/or cosmetic indication to be treated. Topical application to the hair, skin and/or mucous membranes is especially preferred, while application to the (facial) skin is most preferred. The frequency of application will depend on the type and severity of the indication. The W/O emulsion is preferably applied once a week to several times daily and especially once a day to several times daily (for example, two or three times daily).

The following examples illustrate the invention:

### EXAMPLE 1

A W/O emulsion in the form of a cream with a pH of 4 was prepared from the following ingredients.

| Cream | |
|---|---|
| polyglyceryl-3-polyricinoleate | 1.50 g |
| sorbitan oleate | 3.50 g |
| cetearyl isononanoate | 6.00 g |
| isohexadecane | 7.00 g |
| dicaprylyl ether | 5.00 g |
| sucrose polystearate | 1.00 g |
| hydrogenated vegetable oil | 1.50 g |
| tocopherol (70%) | 0.10 g |
| water, purified | 69.56 g |
| glycolic acid (70%) | 1.43 g |
| ammonium hydroxide (25%) | 0.61 g |
| glycerol (85%) | 2.00 g |
| magnesium sulfate | 0.50 g |
| perfume | 0.30 g |
| total | 100.00 g |

The composition was prepared as follows: the ingredients of the oil phase including the emulsifier system (i.e., polyglyceryl-3-polyricinoleate, sorbitan oleate, cetearyl isononanoate, isohexadecane, dicaprylyl ether, sucrose polystearate, hydrogenated vegetable oil, tocopherol (70%)) were then placed in a heatable container, heated to 85° C., melted and mixed. The remaining ingredients, except for the perfume, were dissolved in purified water and the aqueous phase was heated to 85° C. The aqueous phase was added to the oil phase, then homogenized and cooled. Finally, the perfume was added at 45° C.

It has been found that the W/O emulsion according to the invention remains stable during preparation and storage despite the low pH of only 4, i.e., no phase separation or precipitation was observed. The emulsion instead remained visually unchanged, which demonstrates the hydrolysis resistance of the emulsifier system according to the invention.

### EXAMPLE 2

A W/O emulsion in the form of a solution with a pH of 4 was prepared from the following ingredients.

| Lotion | |
|---|---|
| polyglyceryl-3-polyricinoleate | 1.50 g |
| sorbitan oleate | 3.50 g |
| ethylhexyl stearate | 8.00 g |
| isohexadecane | 5.00 g |
| decyloleate | 6.00 g |
| sucrose polystearate | 1.00 g |
| tocopherol (70%) | 0.10 g |
| water, purified | 66.86 g |
| glycolic acid (70%) | 1.43 g |
| ammonium hydroxide (25%) | 0.61 g |
| glycerol (85%) | 5.00 g |
| magnesium sulfate | 0.70 g |
| perfume | 0.30 g |
| total | 100.00 g |

Preparation took place as follows: the ingredients of the oil phase including the emulsifier system (i.e., polyglyceryl-3-polyricinoleate, sorbitan oleate, ethylhexyl stearate, isohexadecane, decyl oleate, sucrose polystearate, tocopherol (70%)) were added and heated at 85° C. in a heatable container, melted and mixed. The remaining ingredients except for the perfume were dissolved in purified water, and the aqueous phase was heated to 85° C. The aqueous phase was added to the oil phase, then the mixture was homogenized and cooled. Finally, the perfume was added at 45° C.

It was found that the W/O emulsion according to the invention would remain stable during preparation and storage despite the low pH of only 4, i.e., no phase separation or precipitation was observed. The emulsion instead remained visually unchanged, which demonstrates the hydrolysis stability of the emulsifier system according to the invention.

### EXAMPLE 3 (COMPARATIVE EXAMPLE)

An O/W emulsion in the form of a cream with a pH of 4 was prepared from the following ingredients.

| Cream | |
|---|---|
| PPG-15-stearyl ether | 4.00 g |
| propylene glycol | 4.00 g |
| steareth-2 | 3.00 g |
| Isohexadecane | 5.00 g |
| steareth-21 | 2.00 g |
| cetearyl alcohol | 1.00 g |
| Dimethicone | 1.00 g |
| Phenoxyethanol | 1.00 g |
| water, purified | 66.00 g |
| glycolic acid (70%) | 10.00 g |
| ammonium hydroxide (25%) | 1.20 g |
| stearic acid | 1.50 g |
| xanthan gum | 0.30 g |
| Total | 100.00 g |

The composition was prepared as follows: the ingredients of the oil phase (i.e., PPG-15-stearyl ether, steareth-2, isohexadecane, steareth-21, cetearyl alcohol, dimethicone, stearic acid) were heated to 70° C. in a heatable container, melted and mixed. The remaining ingredients were dissolved or swollen (xanthan) in purified water and the aqueous phase was heated to 70° C. The fat phase was added to the aqueous phase, then homogenized and cooled.

As expected, it was found that the O/W emulsion remained stable during preparation and storage, i.e., no phase separation or precipitation was observed.

### EXAMPLE 4: KINETIC STUDIES

The W/O emulsion of Example 2 and the O/W emulsion of Eample 3 were subjected to a kinetic study which included 12 volunteers with an average age of 65 years (±5 years) who would apply the two test emulsions to the insides of their forearms in an amount of 40 □L each on an area of approx. 20 cm² each, distributing the test emulsion with a finger. Then the pH of the skin was measured (B: treatment with Eample 2; C: treatment with example 3; A: untreated skin) using a skin pH meter pH 900 PC (Courage & Khazaka, Cologne, Germany) as a function of time. The pH of the untreated skin was used for comparison.

The results are presented in Table 1, which follows:

TABLE 1

| Time | Treatment | pH | □ pH |
|---|---|---|---|
| before application | A | 4.64 | — |
| | B | 4.64 | — |
| | C | 4.58 | — |
| after 30-60 minutes | A | 4.18 | −0.46 |
| | B | 3.62 | −1.02 |
| | C | 4.13 | −0.45 |
| after 2 hours | A | 4.35 | −0.29 |
| | B | 3.75 | −0.89 |
| | C | 3.69 | −0.89 |
| after 4 hours | A | 4.51 | −0.13 |
| | B | 3.92 | −0.72 |
| | C | 3.60 | −0.98 |
| after 6 hours | A | 4.50 | −0.14 |
| | B | 3.98 | −0.66 |
| | C | 3.63 | −0.95 |
| after 8 hours | A | 4.43 | −0.22 |
| | B | 4.00 | −0.64 |
| | C | 3.56 | −1.02 |

Treatments B and C both led to a definite reduction in the pH of the skin in comparison with the untreated skin A after 2, 4, 6 and 8 hours; in the case of treatment B (according to the invention; Example 2), the same result was already measured after 30-60 minutes. However, when treatment B (according to the invention; Example 2) was compared with treatment C (not according to the invention; Example 3), a much more rapid reduction in the pH was found in the case of the inventive treatment B, which is surprising. Exactly the opposite would be assumed because the O/W emulsion of Example 3 is capable of releasing the protons (H⁺) from the external/continuous aqueous phase, while the W/O emulsion of Example 2 according to the invention must release the protons from the internal/disperse phase. This result is even more surprising since the acid concentration in the O/W emulsion of Example 3 is higher by a factor of 7.

In summary, it can surprisingly be concluded that the W/O emulsions according to the invention not only have an excellent stability at a low pH but also are capable of quickly and definitely reducing the pH of skin and doing so unexpectedly more rapidly than a corresponding O/W emulsion. The W/O emulsions according to the invention are thus capable of restoring the age-related or disease-related disturbed skin functions rapidly and effectively and are consequently excellently suited for medical and cosmetic treatment of the skin. These effects can be attributed to the specific emulsifier system used in the W/O emulsions according to the invention, such that this emulsifier system surprisingly manifests its effect even in the low concentrations and ratios of the three components that are preferably used.

1. A water-in-oil emulsion comprising
a) a disperse/internal aqueous phase comprising an α-hydroxy-C₂-C₆-carboxylic acid;
b) a continuous/external oil phase and)
c) an emulsifier system comprising polyglyceryl-3-polyricinoleate, sorbitan oleate and sucrose polystearate,
wherein the emulsion has a pH of 1 to 5.

2. The water-in-oil emulsion according to claim 1, wherein the α-hydroxy-C₂-C₆-carboxylic acid is selected from the group consisting of glycolic acid; lactic acid, tartaric acid, citric acid, ascorbic acid, malic acid and mixtures thereof.

3. The water-in-oil emulsion according to claim 1, wherein the emulsion has a pH of 2.5 to 4.5.

4. The water-in-oil emulsion according to claim 1, additionally comprising a basic pH-adjusting agent.

5. The water-in-oil emulsion according to claim 1, wherein the oil phase comprises isohexadecane.

6. The water-in-oil emulsion according to claim 1, wherein the emulsion contains 0.5 to 3.0 wt % α-hydroxy-C₂-C₆-carboxylic acid, based on the total weight of the emulsion.

7. The water-in-oil emulsion according to claim 1, wherein the emulsion contains 1.0 to 5.0 wt % polyglyceryl-3-polyricinoleate, 2.0 to 10.0 wt % sorbitan oleate and 0.5 to 3.0 wt % sucrose polystearate, based on the total weight of the emulsion.

8. The water-in-oil emulsion according to claim 1, wherein the emulsion contains 1.0 to 2.0 wt % polyglyceryl 3-polricinoleate, 3.0 to 5.0 wt % sorbitan oleate and 1.0 to 1.5 wt % sucrose polystearate, based on the total weight of the emulsion.

9. The water-in-oil emulsion according to claim 1, wherein the emulsion contains 1.0 to 1.5 wt % polyglyceryl-3-polyricinoleate, 3.0 to 3.5 wt % sorhitan oleate and 1.0 to 1.5 wt % sucrose polystearate, based on the total weight of the emulsion.

10. The water-in-oil emulsion according to claim 1, wherein the weight ratio of polmlyceryl-3-polyricinoleote to sorbitan oleate to sucrose polystearate in the emulsifier system is (1.0-1.5) to (3.0-3.5) to (1.0-1.5).

11. The water-in-oil emulsion according to claim 1 for use as a medicament.

12. The water-in-oil emulsion for use according to claim 11 for therapeutic and/or or prophylactic treatment of skin diseases selected from a group consisting of microbial skin infections, skin inflammations, raw skin, dry skin, irritated skin, itching, pruritus, allergies, psoriasis, psoriatic arthritis, eczema, scleroderma, atopic dermatitis, contact dermatitis, systemic lupus erythematosus, alopecia areata, acne and susceptibility to contact allergies.

13. Non-therapeutic use of the water-in-oil emulsion according to claim 1 as a cosmetic.

14. The non-therapeutic use according to claim 13 for improving the epidermal barrier functions and the epidermal barrier integrity, for improving the appearance of skin and/or for increasing the moisture content of skin.

15. The non-therapeutic use according to claim 13 for treatment of cosmetic conditions of the skin, selected from the group consisting of raw skin, dry skin, skin irritations, itching and pruritus, and for preventing skin infections and susceptibility to contact allergies.

16. The water-in-oil emulsion according to claim 2, wherein the α-hydroxy-C₂-C₆-carboxylic acid is glycolic acid, wherein the emulsion has a pH of 3.5 to 4.5.

17. The water emulsion according to claim 4, wherein the pH-adjusting agent is ammonium hydroxide.

18. The water emulsion according to claim 6, wherein the emulsion contains 1.0 to 2.0 wt % α-hydroxy-C₂-C₆-carboxylic acid, based on the total weight of the emulsion.

19. The water-in-oil emulsion according to claim 10, wherein the weight ratio of polyglyceryl-3-polyricinoleate to sorbitan oleate to sucrose polystearate in the emulsifier system is 1.5 to 3.5 to 1.0.

20. The non-therapeutic use of the water-in-oil emulsion according to claim 13 as a cosmetic for restoring the physiological pH of skin.
